(19) United States
(12) Patent Application Publication
(10) Pub. No.: US 2025/0243141 A1
(43) Pub. Date: Jul. 31, 2025

(54) PREPARATION METHOD OF SECONDARY ALCOHOL SURFACE ACTIVE AGENTS

(71) Applicant: Jiangsu Secol Chemical Co.,Ltd, Lianyungang (CN)

(72) Inventor: Dichuan Li, Lianyungang (CN)

(21) Appl. No.: 18/817,140

(22) Filed: Aug. 27, 2024

(30) Foreign Application Priority Data
Jan. 30, 2024 (CN) 202410125331.7

(51) Int. Cl. C07C 29/32; C07C 29/48

(52) U.S. Cl. CPC C07C 29/32; C07C 29/48

(57) ABSTRACT

This invention relates to the preparation method of secondary alcohol surface active agents, which are obtained through two-step alkoxylation of secondary alcohols with diol content of 0.5%-5%. The said secondary alcohols are prepared by the following method. N-alkanes and boron oxygen compounds are added to a reactor for oxidation reaction, after which they are separated through flash distillation and treated by alkali before distillation process during which the secondary alcohols containing 0.5%-5% diols (formed in the oxidation process) are extracted from the top of the column. Diols accounting for 0.5%-5% of the total amount of secondary alcohols are used in this invention as the raw material to significantly improve pour point, gel range and emulsifying power of the final nonionic surface active agent products without affecting their color and odor.

### CROSS-REFERENCE TO RELATED APPLICATIONS

This application claims priority of Chinese Patent Application No. 202410125531.7, filed on Jan. 30, 2024, the entire contents of which are incorporated herein by reference.

### TECHNICAL FIELD

The invention relates to the preparation method of surface active agents, especially secondary alcohol surface active agents.

### TECHNICAL BACKGROUND

Secondary alcohol nonionic surface active agent, Secondary Alcohol Ethoxylate refers to the product obtained by the following method that regular liquid normal paraffin having 8-16 carbon atoms (the mixture of any homologues with no more than 3 consecutive carbon atom numbers, such as the mixture having 12-14 carbon atoms, hereinafter referred to as regular liquid paraffin or n-alkanes) is catalyzed and oxidized to obtain secondary alcohols having 8-16 carbon atoms (the mixture of any homologues with no more than 3 consecutive carbon atom numbers, such as the mixture having 12-14 carbon atoms, hereinafter referred to as diols) and then two-step alkoxylation of secondary alcohols having 8-16 carbon atoms and ethylene oxide (or propylene oxide or/and butylene oxide) is carried out to obtain such product.

In traditional production of secondary alcohol surface active agents, diols are viewed as impurity, which have an adverse influence on the color and odor of the final product, so they are removed in the distillation process. In view of the above problems, it is important to produce secondary alcohols containing a minimum amount of diols so as not to have an obvious impact on the color and odor of the final product. However, by eliminating diol contents in the secondary alcohols, performance parameters such as pour point and gel range in the final alkoxylates are largely compromised. To avoid the dilemma, dedicated research has been carried out and a method of including diol compounds in the secondary alcohol surfactants to enhance their performance without compromising color and odor has been found.

### SUMMARY OF THE INVENTION

The present invention aims to provide secondary alcohol surface active agents, and their preparation method and composition thereof, and wherein the secondary alcohols contain 0.5%-5% of diols, thus significantly improving the pour point, gel range and emulsifying power of the final nonionic surface active agent product without compromising their color and odor.

The present invention adopts the following technical schemes.

The present invention relates to the preparation method of secondary alcohol surface active agents, wherein the said secondary alcohol surface active agent is obtained through two-step alkoxylation of secondary alcohols with diol content of 0.5%-5% and alkoxylates (recycled);

Wherein that molecular formula of the said secondary alcohols are:

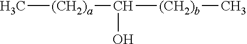

Where a+b=5-13, a≥0 and b≥0;

Wherein, the molecular formula of the diols are:

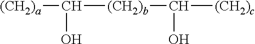

Where a+b+c=6˜14, a≥1, b≥0 and c≥1;

The said alkoxylate is formed by mixing one or more substances among ethylene oxide, propylene oxide and butylene oxide in any proportion.

The present invention relates to preparation method of secondary alcohol surface active agents. Its further optimized scheme is that the secondary alcohol containing 0.5%-5% diols as the raw material is prepared by the following methods:

N-alkanes and boron oxygen compounds are added to a reactor for oxidation reaction, after which they are separated through flash distillation and treated by alkali before the distillation process. During the distillation process, the steam consumption of the reboiler and the reflux ratio at the top of the column are controlled. The reflux ratio at the top of the column is set to be 0.2-3, the temperature at the bottom of the column is set to be 180-200° C., the temperature at the top of the column is set to be 90-120° C., and the pressure at the bottom of the column is set to be 1-10 mmHg. After distillation, the secondary alcohol product is extracted from the top of the column, and the mass content of diols in the secondary alcohol products is 0.5%-5%;

The said boron oxygen compounds are formed by mixing one or more substances among boric acid, metaboric acid and boron oxide in any proportion. The said n-alkanes are mixtures of saturated aliphatic hydrocarbons having 8-16 carbon atoms, preferably 12-14 carbon atoms.

The present invention relates to preparation method of secondary alcohol surface active agents. Its further optimized scheme is that the secondary alcohols containing 0.5%-5% diols as the raw material is prepared by the following methods:

N-alkanes and boron oxygen compounds with the mass ratio of 1,000:70-80 are added to a reactor with an orifice-plate distributor. Mixed gas with oxygen concentration of 5 vol % and nitrogen concentration of 95 vol % is blown to the distributor at a rate of 25 L/h, to react for 2 h at 175° C. under normal atmospheric pressure;

The resulting oxidation reaction mixture is subjected to flash distillation under the atmospheric pressure of 0.6 kPa (A) and at 170° C. to remove unreacted saturated aliphatic hydrocarbons. Thereafter the water is added to the liquid phase at the bottom of the column for hydrolysis at 90° C., to separate the oil phase containing alcohol as well as a small amount of organic acids and organic esters. Next, the oil layer is purified by removing organic acids and organic esters by saponification and washing with water. 5 wt. % NaOH is added to the resulting oil phase for alkali treatment, stirred at 100° C. for 1 h, and transferred to a distillation kettle after cooling for distilling. Under the condition that the pressure at the top of the column is set to be 0.6 kPa (A), the pressure at the bottom of the column is set to be 1.3 kPa (A), the extraction temperature at the top of the column is set to be 90-120° C., and the reflux ratio at the top of the column is set to be 0.2-3, the secondary alcohols with the diol content of 0.5%-5% are extracted.

The present invention relates to preparation method of secondary alcohol surface active agents. Its further optimized scheme is that the mixture of saturated aliphatic hydrocarbons having 12-14 carbon atoms comprises saturated aliphatic hydrocarbon having 12 carbon atoms accounting for 15-30% by mass fraction, the one having 13 carbon atoms accounting for 40-60% by mass fraction and the one having 14 carbon atoms accounting for 20-35% by mass fraction.

The present invention relates to preparation method of secondary alcohol surface active agents. Its further optimized scheme is that the mixture of saturated aliphatic hydrocarbons having 12-14 carbon atoms comprises saturated aliphatic hydrocarbon having 12 carbon atoms accounting for 20-30% by mass fraction, the one having 13 carbon atoms accounting for 40-60% by mass fraction and the one having 14 carbon atoms accounting for 20-30% by mass fraction.

The present invention relates to preparation method of secondary alcohol surface active agents. Its further optimized scheme is that the mixture of saturated aliphatic hydrocarbons having 12-14 carbon atoms comprises saturated aliphatic hydrocarbon having 12 carbon atoms accounting for 22-27% by mass fraction, the one having 13 carbon atoms accounting for 45-55% by mass fraction and the one having 14 carbon atoms accounting for 23-28% by mass fraction.

The present invention relates to preparation method of secondary alcohol surface active agents. Its further optimized scheme is that continuous distillation or batch distillation is used as the said distillation method, and that packed column or plate column is used as the distillation column.

The present invention relates to preparation method of secondary alcohol surface active agents. Its further optimized scheme is that the said two-step addition method comprises the following steps:

Step 1: Alkoxylation of secondary alcohols containing 0.5%-5% diols, catalyst BF3 or its etherate and alkoxylate is carried out to obtain an alkoxylated product with an average adduct number of 0.5-2.5 moles. After catalyst removal and distillation, an alkoxylated intermediate product with an average adduct number of 1-3.5 moles without free alcohol can be obtained. During the distillation, the monohydric alcohol content of secondary alcohol extracted from the column top is 60-95%, and the free secondary alcohol content of alkoxylated intermediate product extracted from the column bottom is 0-1%, which is controlled by controlling steam consumption of reboiler and the reflux ratio at the column top. Thereinto, the reflux ratio at the top of the column is set to be 0.2-3, the temperature at the bottom of the column is set to be 180-200° C., the temperature at the top of the column is set to be 90-120° C., and the pressure is set to be 1-10 mmHg.

Step 2: Alkoxylation of the alkoxylated intermediate product obtained in Step 1 and the alkoxylate is carried out, under the catalysis of an aqueous solution of sodium hydroxide or potassium hydroxide with mass concentration of 30-100% of the alkaline catalyst, to obtain the secondary alcohol surface active agents.

The present invention relates to preparation method of secondary alcohol surface active agents. Its further optimized scheme is that the said two-step addition method comprises the following steps:

Step 1: The secondary alcohols containing 0.5%-5% diols as raw material are added to an autoclave with a stirring device and a temperature control device. Catalyst BF3 or its etherate is added to the autoclave. Ethylene oxide, propylene oxide or butylene oxide are added at a constant rate, under an initial nitrogen pressure of 0.05 Mpa and at 50° C. The mass ratio of secondary alcohol as raw material to alkoxylate is 1,000:310-350 and the added catalyst BF3 or its etherate is 3% % of the secondary alcohol.

After the reaction, the reaction solution is neutralized with 0.5% NaOH aqueous solution at 90° C., and then the solution is washed until its pH value reaches 7. The oil phase is distilled under the pressure of 0.6 kPa (A) and the temperature at the bottom of the column is set to be 180-200° C. to obtain an ethoxylate with an average adduct number of alkoxylates of 3 moles.

Step 2: The resulting ethoxylate is added to the autoclave and then add NaOH. Ethylene oxide, propylene oxide or butylene oxide are added at a constant rate, under an initial nitrogen pressure of 0.05 Mpa and at 150° C. The mass ratio of the alkoxylate added in Step 2 to the secondary alcohol added in Step 1 is 390-410:1,000. After the reaction, it is neutralized with acetic acid to obtain surface active agents as an addition product of ethylene oxide. The average adduct number of the alkoxylates is determined to be 9.0 moles by hydroxyl value measurement.

In this invention, glycol ether is added to the secondary alcohol surface active agents, and wherein the glycol ether and the secondary alcohol surface active agents have the same average adduct number of alkoxylate.

Wherein, the molecular formula of the glycol is:

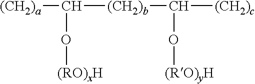

In the formula, R and R′ represent alkylenes having 2-4 carbon atoms respectively, a+b+c=6-14, a≥1, b≥0, c≥1 and x+y=1˜50.

The reaction equation of glycol is as follows:

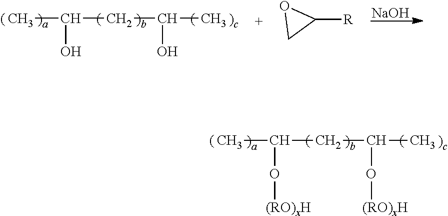

Compared with the prior art, the present invention has the following advantages:

1. In this invention, secondary alcohols with diols accounting for 0.5-5% of its total amount are used as the raw material, to significantly improve the pour point, gel range and emulsifying power of the final nonionic surface active agent product without affecting the color and odor of the final product.

2. This invention limits the temperature at the top of column to 90-120° C. and the pressure at the top of the column to 1-10 mmHg by changing the steam consumption of the reboiler and the reflux flow at the top of the de-heavy distillation column, so as to control the diol content in the secondary alcohol products.

3. The present invention adds raw materials to the secondary alcohol surface active agents to generate glycol ether, and wherein the glycol ether has the same average adduct number of ethylene oxide (or propylene oxide or/and butylene oxide) as the secondary alcohol surface active agent composition containing it. Secondary alcohol nonionic surface active agent products containing glycol ether have the advantages of low pour point, strong fluidity and narrow gel range. The gel performance of the product can be improved and the emulsifying property can be enhanced without affecting the color and odor of the final product.

### DETAILED DESCRIPTION

The present invention is described in details through specific embodiments.

Embodiment 1: preparation method of secondary alcohol surface active agents, with the following steps:

Step 1: oxidation reaction

A reactor with an orifice-plate distributor was charged with 1,000 g of n-alkanes and 75 g of boron oxygen compounds. Subsequently, gas with 5 vol % oxygen concentration and 95 vol % nitrogen concentration was blown to the reactor at a rate of 25 L/h, to react for 2 h at 175° C., under normal pressure.

The resulting oxidation reaction mixture was subjected to flash distillation under the atmospheric pressure of 0.6 kPa (A) and at 170° C. to remove unreacted saturated aliphatic hydrocarbons. Thereafter, 400 g of water was added to the liquid phase at the bottom of column for hydrolysis at 90° C. to separate the oil phase containing alcohol as well as a small amount of organic acids and organic esters. Next, the oil layer was purified by removing organic acids and organic esters by saponification and washing with water.

The 5 wt. % NaOH was added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-120° C., and the reflux ratio at the top of the column was set to be 0.2-3, the secondary alcohol with the diol content of 0.5%-5% was extracted.

Step 2: A two-step ethoxylation process

A 2 L autoclave with a stirring device, a temperature control device and an introduction pipe for adding alkoxylates was charged with 1 kg of the secondary alcohol mixture obtained in Step 1. Thereafter 3 g of BF3 or its etherate catalyst was added to the autoclave, and 330 g of ethylene oxide was blown to the autoclave at a constant rate, under the initial nitrogen pressure of 0.05 MPa and at 50° C.;

After the reaction, the reaction solution was neutralized with 0.5% NaOH aqueous solution at 90° C., and then the solution was washed until its pH value reaches 7. The oil phase was distilled under the pressure of 0.6 kPa (A) and the temperature at the bottom of the column was set to be 180-200° C. to obtain an ethoxylate addition product with an average adduct number of alkoxylates of 3 moles;

Thereafter 500 g of the resulting ethoxylate addition product and 2 g of NaOH were added to the same autoclave, and 398 g of ethylene oxide was added at a constant rate, under the initial nitrogen pressure of 0.05 Mpa and at 150° C. After the reaction, it was neutralized with acetic acid to obtain surface active agents as an addition product of ethylene oxide. The average adduct number of the alkoxylates was determined to be 9.0 moles by hydroxyl value measurement.

Embodiment 2: comparative experiment

The gelation temperature and emulsifying property of the secondary alcohol surface active agents were tested based on the embodiment and comparative examples. The methods of the present invention were adopted by the following experimental examples, and the prior art was used for comparison.

Experimental example 1: preparation method of secondary alcohol surface active agents

### (1) Oxidation Process

A reactor with an orifice-plate distributor was charged with 1,000 g of mixture of saturated aliphatic hydrocarbons having 12-14 carbon atoms (in which the saturated aliphatic hydrocarbons having 12 carbon atoms, 13 carbon atoms and 14 carbon atoms account for 25%, 50% and 25% by mass fraction respectively) and 75 g of metaboric acid to. Gas with an oxygen concentration of 5 vol % and a nitrogen concentration of 95 vol % was blown in at a rate of 25 L/h, to react for 2 h under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter, 400 g of water was added to the liquid phase at the bottom of the column for hydrolysis at 90° C. to separate the oil phase containing alcohol as well as a small amount of organic acids and organic esters. Next, the oil layer was purified by removing organic acids and organic esters by saponification and washing with water. 5 wt. % NaOH was added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-100° C., and the reflux ratio at the top of the column was controlled to be 1:3, the secondary alcohol with the diol content of 0.5% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards, 330 g of ethylene oxide was added to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Experimental example 2: preparation method of secondary alcohol surface active agents

### (1) Oxidation Process

A reactor with an orifice-plate distributor was charged with 1,000 g of a mixture of saturated aliphatic hydrocarbons having 12-14 carbon atoms (in which the saturated aliphatic hydrocarbons having 12 carbon atoms, 13 carbon atoms and 14 carbon atoms account for 30%, 40% and 30% by mass fraction respectively) and 75 g of metaboric acid. Gas with an oxygen concentration of 5 vol % and a nitrogen concentration of 95 vol % was blown in at a rate of 25 L/h, to react for 2 h under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter 400 g of water was added to the liquid phase at the bottom of the column for hydrolysis at 90° C. to separate the oil phase containing alcohol as well as a small amount of organic acids and organic esters. Subsequently, the oil layer was purified by removing organic acids and organic esters by saponification and washing with water. 5 wt. % NaOH was added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-104° C., and the reflux ratio at the top of the column was controlled to be 0.2-3, the secondary alcohol with the diol content of 1% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards, 330 g of ethylene oxide was blown to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Experimental example 3: preparation method of secondary alcohol surface active agents

### (1) Oxidation Process

A reactor with an orifice-plate distributor was charged with 1,000 g of mixture of saturated aliphatic hydrocarbons having 12-14 carbon atoms (in which the saturated aliphatic hydrocarbons having 12 carbon atoms, 13 carbon atoms and 14 carbon atoms account for 20%, 50% and 30% by mass fraction respectively) and 75 g of metaboric acid. Gas with an oxygen concentration of 5 vol % and a nitrogen concentration of 95 vol % was blown in at a rate of 25 L/h, to react for 2 h under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter 400 g of water was added to the liquid phase at the bottom of the column for hydrolysis at 90° C. to separate the oil phase containing alcohol as well as a small amount of organic acids and organic esters. Next, the oil layer was purified by removing organic acids and organic esters by saponification and washing with water. The 5 wt. % NaOH was added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-110° C., and the reflux ratio at the top of the column was controlled to be 0.2-3, the secondary alcohol with the diol content of 2% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards, 330 g of ethylene oxide was blown to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Experimental example 4: preparation method of secondary alcohol surface active agents:

### (1) Oxidation Process

A reactor with an orifice-plate distributor was charged with 1,000 g of a mixture of saturated aliphatic hydrocarbons having 12-14 carbon atoms (in which the saturated aliphatic hydrocarbons having 12 carbon atoms, 13 carbon atoms and 14 carbon atoms account for 30%, 50% and 20% by mass fraction respectively) and 75 g of metaboric acid. The gas with an oxygen concentration of 5 vol % and a nitrogen concentration of 95 vol % was blown in at a rate of 25 L/h, to react for 2 h under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter 400 g of water was added to the liquid phase at the bottom of column for hydrolysis at 90° C. to separate the oil phase containing alcohol as well as a small amount of organic acids and organic esters. Next, the oil layer was purified by removing organic acids and organic esters by saponification and washing with water. The 5 wt. % NaOH is added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-115° C., and the reflux ratio at the top of the column was controlled to be 0.2-3, the secondary alcohol with the diol content of 3% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards, 330 g of ethylene oxide was blown to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Experimental example 5: preparation method of secondary alcohol surface active agents:

### (1) Oxidation Process

A reactor with an orifice-plate distributor was charged with 1,000 g of mixture of saturated aliphatic hydrocarbons having 12-14 carbon atoms (in which the saturated aliphatic hydrocarbons having 12 carbon atoms, 13 carbon atoms and 14 carbon atoms account for 22%, 50% and 28% by mass fraction respectively) and 75 g of metaboric acid. Gas with an oxygen concentration of 5 vol % and a nitrogen concentration of 95 vol % was blown at a rate of 25 L/h, to react for 2 h under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter 400 g of water was added to the liquid phase at the bottom of the column for hydrolysis at 90° C. to separate the oil phase containing alcohol as well as a small amount of organic acids and organic esters. Next, the oil layer was purified by removing organic acids and organic esters by saponification and washing with water. The 5 wt. % NaOH is added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-118° C., and the reflux ratio at the top of the column was controlled to be 0.2-3, the secondary alcohol with the diol content of 4% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards 330 g of ethylene oxide was blown to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Experimental example 6: preparation method of secondary alcohol surface active agents:

### (1) Oxidation Process

A reactor with an orifice-plate distributor was charged with 1,000 g of mixture of saturated aliphatic hydrocarbons having 12-14 carbon atoms (in which the saturated aliphatic hydrocarbons having 12 carbon atoms, 13 carbon atoms and 14 carbon atoms account for 27%, 50% and 23% by mass fraction respectively) and 75 g of metaboric acid. Gas with an oxygen concentration of 5 vol % and a nitrogen concentration of 95 vol % was blown in at a rate of 25 L/h, to react for 2 h and under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter 400 g of water was added to the liquid phase at the bottom of the column for hydrolysis at 90° C. to separate the oil phase containing alcohol as well as a small amount of organic acids and organic esters. Next, the oil layer was purified by removing organic acids and organic esters by saponification and washing with water. The 5 wt. % NaOH is added to the resulting oil phase, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-120° C., and the reflux ratio at the top of the column was controlled to be 0.2-3, the secondary alcohol with the diol content of 5% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards, 330 g of ethylene oxide was blown to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Comparative experiment 1: preparation method of secondary alcohol surface active agents:

### (1) Oxidation Process

A reactor with an orifice-plate distributor was charged with 1,000 g of mixture of saturated aliphatic hydrocarbons with carbon atom numbers of C12: C13: C14=1:2:1 and 75 g of metaboric acid. Gas with an oxygen concentration of 5 vol % and a nitrogen concentration of 95 vol % was blown at a rate of 25 L/h to react for 2 h under normal atmospheric pressure and at 175° C.

The resulting oxidation reaction mixture was subjected to flash distillation under the atmospheric pressure of 0.6 kPa (A) and at 170° C. to remove unreacted saturated aliphatic hydrocarbons. Thereafter 400 g of water was added to the liquid phase at the bottom of the column for hydrolysis at 90° C. to separate the oil phase containing alcohol as well as a small amount of organic acids and organic esters. Next, the oil layer was purified by removing organic acids and organic esters by saponification and washing with water. The 5 wt. % NaOH was added to the resulting oil phase for alkali treatment, stirred at 100° C. for 1 h, and transferred to a distillation kettle after cooling for distilling. Under the condition that the pressure at the top of the column was set to be 0.6 kPa (A), the pressure at the bottom of the column was set to be 1.3 kPa (A), the extraction temperature at the top of the column was set to be 90-98° C., and the reflux ratio at the top of the column was controlled to be 0.2-3, the secondary alcohol with the diol content of 0% is extracted. The resulting secondary alcohol mixture was used as the raw material for alkoxylation of ethylene oxide, and its composition is shown in Table 1.

### (2) Ethoxylation Process

A 2 L autoclave with a stirring device, a temperature control device and an introduction pipe for introducing ethylene oxide (EO) was charged with 1 kg of above secondary alcohol mixture. Subsequently, 3 g of BF3 or its etherate catalyst was added to autoclave. Afterwards, 330 g of ethylene oxide was added to autoclave at a constant rate, under an initial nitrogen pressure of 0.05 MPa and at 50° C. After the reaction, the reaction solution was neutralized with 0.5% NaOH aqueous solution at 90° C. Thereafter the solution was washed until its pH value reaches 7. The oil phase was distilled under the pressure of 0.6 kPa (A) and the temperature at the bottom of the column was set to be 180-200° C. to obtain an ethoxylate addition product with an average adduct number of EO of 3 moles. The same autoclave was charged with 500 g of the resulting ethoxylate addition product and 2 g of NaOH. Afterwards, 398 g of ethylene oxide was blown in at a constant rate, under an initial nitrogen pressure of 0.05 Mpa and at 150° C. After the reaction, it was neutralized with acetic acid to obtain surface active agents as an addition product of ethylene oxide. The average adduct number of EO was determined to be 9.0 moles by hydroxyl value measurement.

Among the above experiments, compared to Embodiment 1, Experiment Examples 2-6 were carried out in the same way as Experiment Example 1, except for the temperature range and reflux ratio at the top of the column for extracting secondary alcohol mixture, and the average adduct number of the resulting secondary alcohol surface active agents was 9.0 moles of EO. In Comparative Experiment 1, the extracted fraction was mainly secondary alcohols with 0% of diols. The distillation operation parameters and composition of the mixture of secondary alcohols for Comparative Example 1 and Experimental Examples 1-6 are shown in Table 1.

TABLE 1
Distillation operation parameters, raw material content and experimental results in Embodiment 2

| | | Raw material | | | | | | | | | |
|---|---|---|---|---|---|---|---|---|---|---|---|
| | | components (wt. %) | | | | | | | | | |
| | Column top | Secondary | | Gelation temperature at | | | | | | Pour | |
| | temperature/ | alcohol | Diol | each concentration/° C. | | | | | | Point | Emulsifying |
| | ° C. | (wt. %) | (wt. %) | 40% | 45% | 50% | 60% | 70% | 80% | (° C.) | property/s |
| Experiment | 90-100 | 98.2 | 0.5 | 25.1 | 29.8 | 46.2 | 44.4 | 42.5 | 20.4 | 11 | 54.3 |
| example 1 | | | | | | | | | | | |
| Experiment | 90-104 | 97.7 | 1 | 23.3 | 28.7 | 28.3 | 26.7 | 31.4 | 20.0 | 10.5 | 59.6 |
| example 2 | | | | | | | | | | | |
| Experiment | 90-110 | 96.8 | 2 | 19.6 | 27.3 | 27.8 | 22.4 | 26.0 | 16.5 | 10.5 | 63.8 |
| example 3 | | | | | | | | | | | |
| Experiment | 90-115 | 95.6 | 3 | <15 | 24.5 | 25.7 | <15 | <15 | <15 | 10 | 68.5 |
| example 4 | | | | | | | | | | | |
| Experiment | 90-118 | 94.6 | 4 | <15 | 22.4 | 23.2 | <15 | <15 | <15 | 9 | 71.5 |
| example 5 | | | | | | | | | | | |
| Experiment | 90-120 | 93.3 | 5 | <15 | <15 | 22.3 | <15 | <15 | <15 | 8 | 70.2 |
| example 6 | | | | | | | | | | | |
| Comparative | 90-98 | 98.7 | 0 | 26.8 | 31.3 | 55.3 | 55.8 | 47.1 | 22.5 | 11 | 51.9 |
| example 1 | | | | | | | | | | | |

Based on the results of gelation and emulsifying properties in Table 1, by combining with Comparative Example 1 and Experimental Examples 1-6, it can be known that the emulsifying property of secondary alcohol surface active agent products using the secondary alcohols containing 0.5%-5% diols as the raw material was between 54.3 and 70.2, and that the higher the diol content, the better the emulsifying and gelation properties, thereby verifying the fact that the product of the present invention has the advantages of low pour point, high fluidity, and narrow gel range, and that the gelation and emulsifying properties of the product are enhanced.

The relevant determination methods in the embodiments are as follow:

### 1. Determination of Gelation Property

Gelation property is determined by the gelation temperature of the surface active agent aqueous solution with a certain concentration, and its determination method is as follows:

Aqueous solutions of surface active agents (25° C.) are prepared with specified concentrations (30 wt %˜80 wt. %) shown in Table 1 and heated to 60° C. Next, the samples are placed in the water bath at 15° C.±0.5° C. with slight shaking. Gelation temperatures are measured visually when a clear flowable liquid turns to a clear or cloudy gel (no fluidity upon pouring). 2. Determination of emulsifying property

Emulsifying property is determined by the time taken for the two-phase separation of emulsion containing oil and surface active agents. The longer the separation time, the better the emulsifying property. The determination method is as follows:

Some 5 g/L aqueous solution of surface active agent compositions are prepared, and then 35 ml aqueous solution of surface active agents and 35 ml oil are weighed in turn with a glass bottle. They are heated in water (oil) bath at 25° C. for 30 min, stirred by magnetic stirrer for 1 min (stirring speed 1,000 r/min), and then left alone for phase separation. The time to separate 20 ml of the water phase at the bottom of each bottle is measured.

The technical features of the above embodiments can be performed or modified by anyone who is familiar with the art. To make the description concise, not all possible combinations of the technical features in the above embodiments are described. However, as long as there is no contradiction in the combinations of these technical features, they should be considered to be within the scope specified in the Instructions.

The above embodiments are only a part of all the embodiments of the present invention, and their descriptions are specific and detailed, but they cannot be understood as the only embodiments of the present invention. It should be pointed out that technical personnel in this field may make variations and improvements to this invention without divorcing from its conception, and these variations and improvements shall be within the scope of protection of the present invention. Therefore, the scope of protection of the present invention patent shall be based on the attached Patent Claims.

1. The preparation method of secondary alcohol surface active agents, featuring that the said secondary alcohol surface active agents are obtained through two-step alkoxylation of secondary alcohols with diol content of 0.5%-5%; 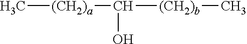
wherein that molecular formula of the secondary alcohols are:
where a+b=5-13, a≥0 and b≥0; 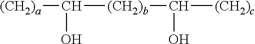
wherein, the molecular formula of the said diols are:
where a+b+c=6˜14, a≥1, b≥0 and c≥1;
the said alkoxylates are formed by mixing one or more substances among ethylene oxide, propylene oxide and butylene oxide in any proportion.

2. The preparation method of secondary alcohol surface active agents of claim 1 wherein the secondary alcohols containing 0.5%-5% diols as the raw material is prepared by the following method:
N-alkanes and boron oxygen compounds are added to a reactor for oxidation reaction, during which secondary alcohols and diols are formed, then they are separated through flash distillation and treated by alkali before the distillation process; during the distillation process, the steam consumption of the reboiler and the reflux ratio at the top of the column are controlled; thereinto, the reflux ratio at the top of the column is set to be 0.2-3, the temperature at the bottom of the column is set to be 180-200° C., the temperature at the top of the column is set to be 90-120° C., and the pressure at the bottom of the column is set to be 1-10 mmHg; after distillation, the secondary alcohol product is extracted from the top of the column, and the mass content of diols in the secondary alcohol products is 0.5%-5%;
the said boron oxygen compounds are formed by mixing one or more substances among boric acid, metaboric acid and boron oxide in any proportion; the said n-alkanes are mixtures of saturated aliphatic hydrocarbons having 8-16 carbon atoms, preferably 12-14 carbon atoms.

3. The preparation method of secondary alcohol surface active agents of claim 1 wherein the secondary alcohols containing 0.5%-5% diols as the raw material is prepared by the following methods:
N-alkanes and boron oxygen compounds with the mass ratio of 1,000:70-80 are added to a reactor with an orifice-plate distributor; mixed gas with oxygen concentration of 5 vol % and nitrogen concentration of 95 vol % is blown to the distributor at a rate of 25 L/h, to react for 2 h at 175° C. under normal atmospheric pressure;
The resulting oxidation reaction mixture is subjected to flash distillation under the atmospheric pressure of 0.6 kPa (A) and at 170° C. to remove unreacted saturated aliphatic hydrocarbons; thereafter water is added to the liquid phase at the bottom of the column for hydrolysis at 90° C., to separate the oil phase containing alcohol as well as a small amount of organic acids and organic esters; next, the oil layer is purified by removing organic acids and organic esters by saponification and washing with water; the 5 wt. % NaOH is added to the resulting oil phase for alkali treatment, stirred at 100° C. for 1 h, and transferred to a distillation kettle after cooling for distilling; under the condition that the pressure at the top of the column is set to be 0.6 kPa (A), the pressure at the bottom of the column is set to be 1.3 kPa (A), the extraction temperature at the top of the column is set to be 90-120° C., and the reflux ratio at the top of the column is set to be 0.2-3, the secondary alcohols with the diol content of 0.5%-5% are extracted.

4. The preparation method of secondary alcohol surface active agents of claim 2 wherein the secondary alcohols containing 0.5%-5% diols as the raw material is prepared by the following methods:
N-alkanes and boron oxygen compounds with the mass ratio of 1,000:70-80 are added to a reactor with an orifice-plate distributor; mixed gas with oxygen concentration of 5 vol % and nitrogen concentration of 95 vol % is blown to the distributor at a rate of 25 L/h, to react for 2 h at 175° C. under normal atmospheric pressure;
The resulting oxidation reaction mixture is subjected to flash distillation under the atmospheric pressure of 0.6 kPa (A) and at 170° C. to remove unreacted saturated aliphatic hydrocarbons; thereafter water is added to the liquid phase at the bottom of the column for hydrolysis at 90° C., to separate the oil phase containing alcohol as well as a small amount of organic acids and organic esters; Next, the oil layer is purified by removing organic acids and organic esters by saponification and washing with water; the 5 wt. % NaOH is added to the resulting oil phase for alkali treatment, stirred at 100° C. for 1 h, and transferred to a distillation kettle after cooling for distilling; under the condition that the pressure at the top of the column is set to be 0.6 kPa (A), the pressure at the bottom of the column is set to be 1.3 kPa (A), the extraction temperature at the top of the column is set to be 90-120° C., and the reflux ratio at the top of the column is set to be 0.2-3, the secondary alcohols with the diol content of 0.5%-5% are extracted.

5. The preparation method of the secondary alcohol surface active agents of claim 2 wherein the mixture of saturated aliphatic hydrocarbons having 12-14 carbon atoms comprises saturated aliphatic hydrocarbon having 12 carbon atoms accounting for 15-30% by mass fraction, the one having 13 carbon atoms accounting for 40-60% by mass fraction and the one having 14 carbon atoms accounting for 20-35%.

6. The preparation method of the secondary alcohol surface active agents of claim 4 wherein the mixture of saturated aliphatic hydrocarbons having 12-14 carbon atoms comprises saturated aliphatic hydrocarbon having 12 carbon atoms accounting for 20-30% by mass fraction, the one having 13 carbon atoms accounting for 40-60% by mass fraction and the one having 14 carbon atoms accounting for 20-30%.

7. The preparation method of the secondary alcohol surface active agents of claim 5 wherein the mixture of saturated aliphatic hydrocarbons having 12-14 carbon atoms comprises saturated aliphatic hydrocarbon having 12 carbon atoms accounting for 22-27% by mass fraction, the one having 13 carbon atoms accounting for 45-55% by mass fraction and the one having 14 carbon atoms accounting for 23-28%.

8. The preparation method of the secondary alcohol surface active agents of claim 2 wherein continuous distillation or batch distillation is used as the said distillation method, and that packed column or plate column is used as the said distillation column.

9. The preparation method of secondary alcohol surface active agents of claim 1 wherein the two-step alkoxylation method comprises the following steps:
step 1: Alkoxylation of secondary alcohol containing 0.5%-5% diols, catalyst BF3 or its etherate and alkoxylates (recycled) is carried out to obtain an alkoxylated product with an average adduct number of 0.5-2.5 moles; after catalyst removal and distillation, an alkoxylated intermediate product with an average adduct number of 1-3.5 moles without free alcohol can be obtained; during the distillation, the monohydric alcohol content of secondary alcohol extracted from the column top is 60-95%, and the free secondary alcohol content of the alkoxylated intermediate product extracted from the column bottom is 0-1%, which is controlled by monitoring the temperature of reboiler and the reflux ratio at the column top; thereinto, the reflux ratio at the top of the column is set to be 0.2-3, the temperature at the bottom of the column is set to be 180-200° C., the temperature at the top of the column is set to be 90-120° C., and the pressure is set to be 1-10 mmHg;
step 2: Alkoxylation of the acid-catalyzed alkoxylated intermediate obtained in Step 1 and EO/PO/BO is further carried out, under the catalysis of an aqueous solution of sodium hydroxide or potassium hydroxide with a mass concentration of 30-100% of the alkaline catalyst, to obtain higher-mole secondary alcohol surface active agents.

10. The preparation method of secondary alcohol surface active agents of claim 2 wherein the two-step alkoxylation method comprises the following steps:
step 1: Alkoxylation of secondary alcohol containing 0.5%-5% diols, catalyst BF3 or its etherate and alkoxylates (recycled) is carried out to obtain an alkoxylated product with an average adduct number of 0.5-2.5 moles; after catalyst removal and distillation, an alkoxylated intermediate product with an average adduct number of 1-3.5 moles without free alcohol can be obtained; during the distillation, the monohydric alcohol content of secondary alcohol extracted from the column top is 60-95%, and the free secondary alcohol content of the alkoxylated intermediate product extracted from the column bottom is 0-1%, which is controlled by monitoring the temperature of reboiler and the reflux ratio at the column top; thereinto, the reflux ratio at the top of the column is set to be 0.2-3, the temperature at the bottom of the column is set to be 180-200° C., the temperature at the top of the column is set to be 90-120° C., and the pressure is set to be 1-10 mmHg;
step 2: Alkoxylation of the acid-catalyzed alkoxylated intermediate obtained in Step 1 and EO/PO/BO is further carried out, under the catalysis of an aqueous solution of sodium hydroxide or potassium hydroxide with a mass concentration of 30-100% of the alkaline catalyst, to obtain higher-mole secondary alcohol surface active agents.

11. The preparation method of secondary alcohol surface active agents of claim 1 wherein the two-step addition method comprises the following steps:
step 1: The secondary alcohols containing 0.5%-5% diols as raw material are added to an autoclave with a stirring and temperature control device; catalyst BF3 or its etherate is added to the autoclave; ethylene oxide, or propylene oxide or butylene oxide are added at a constant rate, under an initial nitrogen pressure of 0.05 Mpa and at 50° C.; the mass ratio of secondary alcohol as raw material to alkoxylate is 1,000:310-350 and the added catalyst BF3 or its etherate is 3% % of the secondary alcohol;
after the reaction, the reaction solution is neutralized with 0.5% NaOH aqueous solution at 50-98° C.; thereafter the solution is washed until its pH value reaches 7; the oil phase is distilled under the pressure of 0.6 kPa (A) and the temperature at the bottom of the column is set to be 180-200° C. to obtain an alkoxylated product with an average adduct number of 3 moles;
step 2: The resulting alkoxylated product is added to the autoclave and then add NaOH Ethylene oxide, propylene oxide or butylene oxide are added at a constant rate, under an initial nitrogen pressure of 0.05 Mpa and at 150° C.; the mass ratio of the alkoxylate added in Step 2 to the secondary alcohol added in Step 1 is 390-410:1,000; after the reaction, it is neutralized with acetic acid to obtain a surface active agents as an addition product of ethylene oxide; the average adduct number of the alkoxylates is determined to be 9.0 moles by hydroxyl value measurement.

12. The preparation method of secondary alcohol surface active agents of claim 2 wherein the two-step addition method comprises the following steps:
step 1: The secondary alcohols containing 0.5%-5% diols as raw material are added to an autoclave with a stirring and temperature control device; catalyst BF3 or its etherate is added to the autoclave; ethylene oxide, or propylene oxide or butylene oxide are added at a constant rate, under an initial nitrogen pressure of 0.05 Mpa and at 50° C.; the mass ratio of secondary alcohol as raw material to alkoxylate is 1,000:310-350 and the added catalyst BF3 or its etherate is 3% % of the secondary alcohol;
after the reaction, the reaction solution is neutralized with 0.5% NaOH aqueous solution at 50-98° C.; thereafter the solution is washed until its pH value reaches 7; the oil phase is distilled under the pressure of 0.6 kPa (A) and the temperature at the bottom of the column is set to be 180-200° C. to obtain an alkoxylated product with an average adduct number of 3 moles;
step 2: The resulting alkoxylated product is added to the autoclave and then add NaOH. Ethylene oxide, propylene oxide or butylene oxide are added at a constant rate, under an initial nitrogen pressure of 0.05 Mpa and at 150° C.; the mass ratio of the alkoxylate added in Step 2 to the secondary alcohol added in Step 1 is 390-410:1,000; after the reaction, it is neutralized with acetic acid to obtain a surface active agents as an addition product of ethylene oxide; the average adduct number of the alkoxylates is determined to be 9.0 moles by hydroxyl value measurement.
